(19) United States
(12) Patent Application Publication
(10) Pub. No.: US 2022/0373249 A1
(43) Pub. Date: Nov. 24, 2022

(54) SECONDARY CONTAINER WITH FLOW-THROUGH APERTURES FOR FREEZING, THAWING, AND SHIPPING PRODUCTS, AND ASSOCIATED METHODS

(71) Applicant: BIOMARIN PHARMACEUTICAL INC., Novato, CA (US)

(72) Inventors: Richard WISNIEWSKI, Novato, CA (US); Saeed MOSHASHAEE, Novato, CA (US); Pooja SANE, Novato, CA (US)

(73) Assignee: BIOMARIN PHARMACEUTICAL INC., Novato, CA (US)

(21) Appl. No.: 17/771,533

(22) PCT Filed: Nov. 13, 2020
(86) PCT No.: PCT/US2020/060512

§ 371 Date: Apr. 25, 2022

Related U.S. Application Data
(60) Provisional application No. 62/935,863, filed on Nov. 15, 2019.

(51) Int. Cl. F25D 25/00; F25D 15/00; F25D 17/06; F25D 3/10; B65D 1/38

(52) U.S. Cl. CPC F25D 25/005; F25D 15/00; F25D 17/06; F25D 3/10; B65D 1/38

(57) ABSTRACT

Containers are provided herein for facilitating freezing, thawing, and/or storage of products that may be perishable, such as pharmaceuticals. The containers can include shells that enclose the product, and that include apertures permitting flow of a fluid through the containers' internal volume. The temperature of the fluid can be selected so as to freeze or thaw the product. The container optionally can include pressure building members that control the flow of the fluid through the containers' inner volume, e.g., increase the fluid flow rate and thus increase the rate with which the fluid is frozen or thawed.

### FIELD

This application relates to secondary containers for products, such as biological or pharmaceutical products.

### BACKGROUND

Certain products, such as certain biological or pharmaceutical products, are highly perishable but can be preserved by maintaining them in a frozen state until an appropriate time, e.g., until immediately before they are to be administered to a subject. It therefore can be useful to ship such products while in a frozen state.

### SUMMARY

Secondary containers with flow-through apertures for freezing, thawing, and shipping products, and associated methods, are provided herein.

Under one aspect, a container is provided herein that includes an upper shell and a lower shell. The upper shell can include an upper surface, a first flange, and a first plurality of sidewalls extending between the upper surface and the first flange. Apertures can be defined through first and second sidewalls of the first plurality of sidewalls. The lower shell can include a lower surface, a second flange, and a second plurality of sidewalls extending between the lower surface and the second flange. Apertures can be defined through first and second sidewalls of the second plurality of sidewalls. The first and second flanges can contact one another to define an internal volume. The apertures defined through the first and second sidewalls of the first plurality of sidewalls and the apertures defined through the first and second sidewalls of the second plurality of sidewalls can be configured such that fluid flows through the internal volume through those apertures.

In some configurations, the upper shell further includes a first pressure building member. Optionally, the lower shell further includes a second pressure building member. Additionally, or alternatively, the first pressure building member optionally increases a rate of flow of the fluid through the internal volume. Optionally, the first pressure building member includes a linear bar disposed outside of the internal volume along the apertures defined through the first sidewall of the first plurality of sidewalls. Additionally, or alternatively, the first pressure building member optionally includes a fence disposed outside of the internal volume around the apertures defined through the first sidewall of the first plurality of sidewalls.

Additionally, or alternatively, the upper shell optionally includes a first support member and the lower shell includes a second support member. The first and second support members can be configured to support an inner container within the internal volume. Optionally, the first and second support members independently are selected from the group consisting of a membrane, a fabric, a strap, a net, a sheet, a film, a foil, a mesh, cables, bands, ribbons, cords, bars, rods, and screens, and any suitable combinations thereof. Additionally, or alternatively, the first and second support members each optionally includes perforations defined therethrough, and wherein the fluid contacts the inner container via the perforations. Additionally, or alternatively, the first and second support members optionally each are elastic. In one example, the first and/or second support members are pre-shaped to accommodate the inner container, for example as two halves of a mesh basket holding the inner container within the internal volume.

Additionally, or alternatively, the first and second sidewalls of the first plurality of sidewalls optionally are disposed parallel to one another. The first and second sidewalls of the second plurality of sidewalls optionally can be disposed parallel to one another and parallel to the first and second sidewalls of the first plurality of sidewalls.

Additionally, or alternatively, the fluid flows through the internal volume at a rate of at least about 1 meter per second. Other exemplary rates of fluid flow include, but are not limited to, at least 0.001 meters per second, or at least 0.005 meters per second, or at least 0.05 meters per second, or at least 0.1 meters per second.

Additionally, or alternatively, the fluid optionally flows through a portion of the internal volume within the upper shell at a rate that is about the same as through a portion of the internal volume within the lower shell.

Additionally, or alternatively, the fluid optionally is a gas.

Additionally, or alternatively, the container optionally includes an inner container disposed within the internal volume. The inner container optionally stores a product. The product optionally includes a frozen solid. The fluid optionally is at a temperature selected to thaw the frozen solid. Additionally, or alternatively, the product optionally includes a liquid. The fluid optionally is at a temperature selected to freeze the liquid. Optionally, the product is perishable.

Additionally, or alternatively, the upper surface, the first plurality of sidewalls, and the first flange are of unitary construction; the lower surface, the second plurality of sidewalls, and the second flange are of unitary construction; and the first and second flanges are separable from one another.

Under another aspect, also provided herein is an assembly including a stack of any such containers.

Under yet another aspect, a kit is provided herein that includes an upper shell and a lower shell. The upper shell can include an upper surface, a first flange, and a first plurality of sidewalls extending between the upper surface and the first flange. Apertures can be defined through first and second sidewalls of the first plurality of sidewalls. The lower shell can include a lower surface, a second flange, and a second plurality of sidewalls extending between the lower surface and the second flange. Apertures can be defined through first and second sidewalls of the second plurality of sidewalls. The first and second flanges can be configured to be brought into contact with one another to define an internal volume.

Under another aspect, a container is provided herein that includes an upper shell and a lower shell. The upper shell includes an upper shell first compartment, an upper shell second compartment, an upper shell flange, and an upper shell junction. The upper shell first compartment includes a plurality of upper shell first compartment walls and an upper shell first compartment floor. Apertures are defined through the upper shell first compartment floor. The upper shell second compartment includes a plurality of upper shell second compartment walls and an upper shell second compartment floor. The upper shell flange extends around the upper shell first compartment and the upper shell second compartment. The upper shell junction extends between and separating the upper shell first compartment from the upper shell second compartment. The lower shell includes a lower shell first compartment, a lower shell second compartment, a lower shell flange, and a lower shell junction. The lower shell first compartment includes a plurality of lower shell first compartment walls and a lower shell first compartment floor Apertures are defined through the lower shell first compartment floor. The lower shell second compartment includes a plurality of lower shell second compartment walls and a lower shell second compartment floor. The lower shell flange extends around the lower shell first compartment and the lower shell second compartment. The lower shell junction extends between and separating the lower shell first compartment from the lower shell second compartment. The upper shell flange and the lower shell flange contact one another to define an internal volume. The apertures defined through the upper shell first compartment floor and the apertures defined through the lower shell first compartment floor are configured such that fluid flows through the internal volume through those apertures.

Additionally, or alternatively, the container further includes one or more pinches positioned attached to at least one of the upper shell junction and the lower shell junction.

Additionally, or alternatively, the container further includes one or more holes and one or more counterpart holes extending through the upper shell flange and the lower shell flange, respectively.

Additionally, or alternatively, the container further includes a fastener. Examples of fasteners include a cable tie, a quarter turn fastener, a spring-loaded fastener, a bolt with a locking nut, a bolt with a winged nut, a clamp, a screw, a rivet, a pin, a wire loop, a snap-in fastener, and a polymer insert.

Additionally, or alternatively, at least one of the upper shell first compartment floor and the lower shell first compartment floor is wavy.

Additionally, or alternatively, at least one of the upper shell second compartment floor and the lower shell second compartment floor is flat.

Additionally, or alternatively, the upper shell first compartment has a greater depth than upper shell second compartment and the lower shell first compartment has a greater depth than the lower shell second compartment.

Additionally, or alternatively, a depth of the upper shell first compartment and the lower shell first compartment is approximately 1.5 inches or 38.1 millimeters and a depth of the upper shell second compartment and the lower shell second compartment is approximately 0.6 inches or 15.24 millimeters.

Additionally, or alternatively, the upper shell flange and the lower shell flange each have four curved corners having a radius of curvature of approximately 1.5 inches or 38.1 millimeters.

Additionally, or alternatively, the apertures are oval in shape and have a width of approximately 0.7 inches or 17.78 millimeters and a length of approximately 1.3 inches or 33.02 millimeters.

Additionally, or alternatively, the fluid flows through the internal volume at a rate of at least about 1 meter per second. Other exemplary rates of fluid flow include, but are not limited to, at least 0.001 meters per second, or at least 0.005 meters per second, or at least 0.05 meters per second, or at least 0.1 meters per second.

Additionally, or alternatively, the fluid optionally flows through a portion of the internal volume within the upper shell at a rate that is about the same as through a portion of the internal volume within the lower shell.

Additionally, or alternatively, the fluid optionally is a gas.

Additionally, or alternatively, the container optionally includes an inner container disposed within the internal volume. The inner container optionally includes a bag, tubing, and an interface between the bag and the tubing. The inner container optionally stores a product. The product optionally includes a frozen solid. The fluid optionally is at a temperature selected to thaw the frozen solid. Additionally, or alternatively, the product optionally includes a liquid. The fluid optionally is at a temperature selected to freeze the liquid. Optionally, the product is perishable. The inner container is optionally disposed between the upper shell first compartment and the lower shell first compartment or between the upper shell second compartment and the lower shell second compartment. The inner container optionally has an interface that is disposed between the upper shell junction and the lower shell junction.

Additionally, or alternatively, the upper shell first compartment, the upper shell second compartment, the upper shell flange, and the upper shell junction are of unitary construction; the lower shell first compartment, the lower shell second compartment, the lower shell flange, and the lower shell junction are of unitary construction; and the upper shell flange and the lower shell flange are separable from one another.

Additionally, or alternatively, the upper shell is identical to the lower shell.

Additionally, or alternatively, the container in response to freezing an inner container disposed within the container, a freezing rate for a top and bottom of the inner container is the same and a freezing profile a top and bottom of the inner container is even.

Additionally, or alternatively, each of the upper shell and the lower shell measures approximately 24.4 inches or 619.76 millimeters by approximately 16 inches or 406.4 millimeters.

Under another aspect, also provided herein is an assembly including a stack of any such containers.

Under another aspect, a kit is provided herein that includes an upper shell and a lower shell. The upper shell includes an upper shell first compartment, an upper shell second compartment, an upper shell flange, and an upper shell junction. The upper shell first compartment includes a plurality of upper shell first compartment walls and an upper shell first compartment floor. Apertures are defined through the upper shell first compartment floor. The upper shell second compartment includes a plurality of upper shell second compartment walls and an upper shell second compartment floor. The upper shell flange extends around the upper shell first compartment and the upper shell second compartment. The upper shell junction extends between and separating the upper shell first compartment from the upper shell second compartment. The lower shell includes a lower shell first compartment, a lower shell second compartment, a lower shell flange, and a lower shell junction. The lower shell first compartment includes a plurality of lower shell first compartment walls and a lower shell first compartment floor Apertures are defined through the lower shell first compartment floor. The lower shell second compartment includes a plurality of lower shell second compartment walls and a lower shell second compartment floor. The lower shell flange extends around the lower shell first compartment and the lower shell second compartment. The lower shell junction extends between and separating the lower shell first compartment from the lower shell second compartment. The upper shell flange and the lower shell flange contact one another to define an internal volume. The apertures defined through the upper shell first compartment floor and the apertures defined through the lower shell first compartment floor are configured such that fluid flows through the internal volume through those apertures.

Under still another aspect, a method of freezing a product is provided herein. The product can be disposed within an inner container. The method can include disposing, within an internal volume of a secondary container, the inner container having the product disposed therein. The method also can include freezing the product by flowing fluid through the internal volume via apertures defined through at least one sidewall of the secondary container, wherein the fluid is at a temperature selected to freeze the product.

Optionally, the method further includes, after freezing the product, storing or shipping the container having the inner container disposed within the internal volume while maintaining the frozen product.

Additionally, or alternatively, the method optionally further includes thawing the frozen product by flowing a second fluid through the internal volume via the apertures, wherein the second fluid is at a temperature selected to thaw the frozen product.

Under yet another aspect, a method of thawing a frozen product is provided. The frozen product can be disposed within an inner container. The method can include providing, within an internal volume of a secondary container, the inner container having the frozen product disposed therein. The method also can include thawing the frozen product by flowing fluid through the internal volume via apertures defined through at least one sidewall of the secondary container, wherein the fluid is at a temperature selected to thaw the frozen product.

Optionally, the method further includes disposing, within the internal volume of the secondary container, the inner container having the product disposed therein. The method further optionally can include freezing the product into a frozen solid by flowing a first fluid through the internal volume via apertures defined through at least one sidewall of the secondary container, wherein the first fluid is at a temperature selected to freeze the product.

Additionally, or alternatively, the method optionally further includes, after freezing the product, storing or shipping the container having the inner container disposed within the internal volume while maintaining the frozen product.

### BRIEF DESCRIPTION OF DRAWINGS

FIGS. 1A and 1B schematically illustrate side views of a secondary container with flow-through apertures, according to an exemplary configuration provided herein.

FIGS. 1C-1D schematically illustrate side views of a secondary container holding an example primary container, according to an exemplary configuration provided herein.

FIGS. 1E-1F schematically illustrate side views of a secondary container holding another example primary container, according to an exemplary configuration provided herein.

FIG. 1G schematically illustrates an exploded view of the secondary container of FIGS. 1C-1D having an exemplary primary container disposed therein, according to an exemplary configuration provided herein.

FIGS. 2A-2B schematically illustrate side views of an upper part of the secondary container, according to an exemplary configuration provided herein.

FIGS. 2C-2D schematically illustrate side views of an upper part of the secondary container, according to another exemplary configuration provided herein.

FIGS. 3A-3B schematically illustrate side views of a lower part of the secondary container, according to an exemplary configuration provided herein.

FIGS. 3C-3D schematically illustrate side views of a lower part of the secondary container, according to another exemplary configuration provided herein.

FIG. 3E schematically illustrates a perspective view of a lower part of the secondary container, according to an exemplary configuration provided herein.

FIGS. 4A-4C schematically illustrate plan views of a lower part of the secondary container including exemplary support members for the primary container, according to exemplary configurations provided herein.

FIG. 5 schematically illustrates a side view of an assembly including a stack of secondary containers with flow-through apertures, according to exemplary configurations provided herein.

FIG. 6 illustrates a flow of operations in an exemplary method for handling a primary container using a secondary container with flow-through apertures, according to exemplary configurations provided herein.

FIGS. 7A-7B schematically illustrate exemplary static pressure distributions around an exemplary secondary container with flow-through apertures, according to one nonlimiting example.

FIGS. 8A-8B are photographs of a demonstration of the effect of pressure building members, according to one nonlimiting example.

FIGS. 9A-9B are photographs of another demonstration of the effect of pressure building members, according to one nonlimiting example.

FIG. 10 schematically illustrates a perspective view of a lower shell of another example of a secondary container with flow-through apertures, according to an exemplary configuration provided herein.

FIG. 11 schematically illustrates a top planar view of the lower shell with flow-through apertures of FIG. 10, according to an exemplary configuration provided herein.

FIG. 12 schematically illustrates a cross-sectional view of the lower shell at the line 12-12 of FIG. 11, according to an exemplary configuration provided herein.

FIG. 13 schematically illustrates a side view of the secondary container including the upper shell and the lower shell, according to an exemplary configuration provided herein.

FIG. 14 schematically illustrates an end view of the upper shell, according to an exemplary configuration provided herein.

FIG. 15 illustrates a top planar view of the lower shell of the secondary container with the pinch and a primary container, according to an exemplary configuration provided herein.

FIG. 16A is a graph illustrating the temperature over time profile of a bag of the primary container without using a secondary container, according to exemplary configurations provided herein.

FIG. 16B is a graph illustrating the temperature over time profile of a bag of the primary container while using a secondary container, according to exemplary configurations provided herein.

FIGS. 17A-17B schematically illustrate side views of assemblies including stacks of secondary containers with flow-through apertures, according to exemplary configurations provided herein.

### DETAILED DESCRIPTION

Secondary containers with flow-through apertures for freezing, thawing, and shipping products, and associated methods, are provided herein. For example, the apertures can enhance the flow of fluid (such as a gas) through the secondary container that in turn improves cooling (e.g., as in freezing) or warming (e.g., as in thawing), or gas or vapor exchange (e.g., as in evaporation, distillation, or drying). In another example, the apertures can enhance the flow of fluid (such as a gas) through the secondary container that in turn uniformly cools (e.g., as in freezing) or warms (e.g., as in thawing), or uniform gas or vapor exchange (e.g., as in evaporation, distillation, or drying).

For example, certain products, such as certain biological or pharmaceutical products, are highly perishable, e.g., can degrade rapidly. The present disclosure provides improved methods of handling such products. For example, such products can be stored in a primary container, such as a bag or vial. The primary container can be disposed within an internal volume of a secondary container that includes apertures facilitating flow of a fluid through that internal volume. Fluid at an appropriate temperature can be flowed through the internal volume via the apertures to rapidly freeze the product, e.g., so as to place the product in a state in which degradation occurs slowly, if at all. While in the frozen state, the product can be stored or shipped, e.g., while within both the primary and secondary containers. At an appropriate time, e.g., before it is intended to administer the product to a subject, the frozen product can be thawed. For example, fluid at an appropriate temperature can be flowed through the internal volume via the apertures can be used to rapidly thaw the product.

Such fluid flow can be based, at least in part, on a static pressure difference between different zones on the wall(s) of the secondary container. For example, the flow of fluid through the secondary container can form high and low static pressure zones on the container wall surface. The fluid can enter the secondary container through apertures in a high static pressure zone and leave the container through apertures in the low static pressure zone. The apertures can be in the high static pressure zone for the fluid entry and can be in the low gas pressure zone for the fluid exit. The fluid exit apertures can be located in the back of the secondary container (distal from the fluid entrance apertures) to allow fluid to flow through the whole secondary container, or substantially the whole secondary container. In some cases, the exit apertures can be located in other zones of low pressure, for example, if a stagnant zone inside the secondary container is desired.

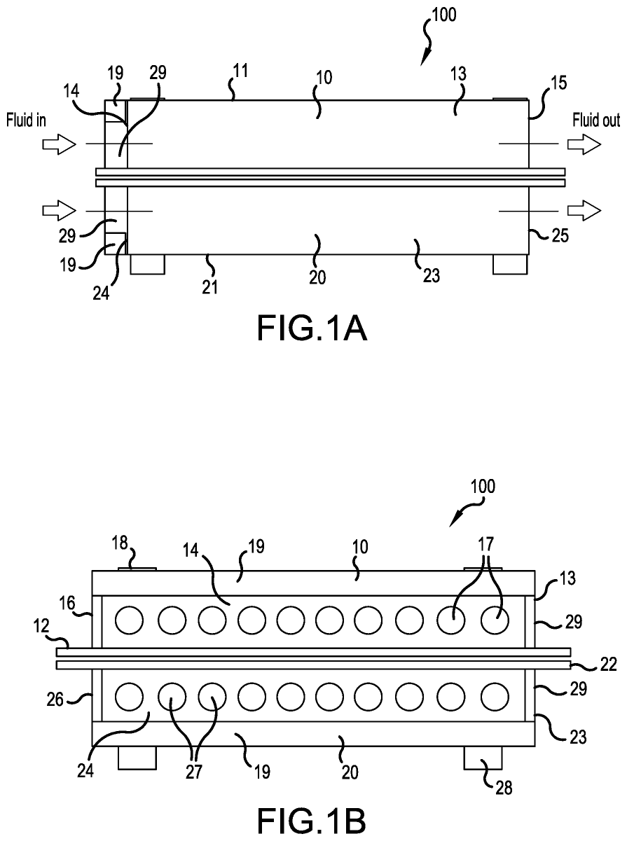

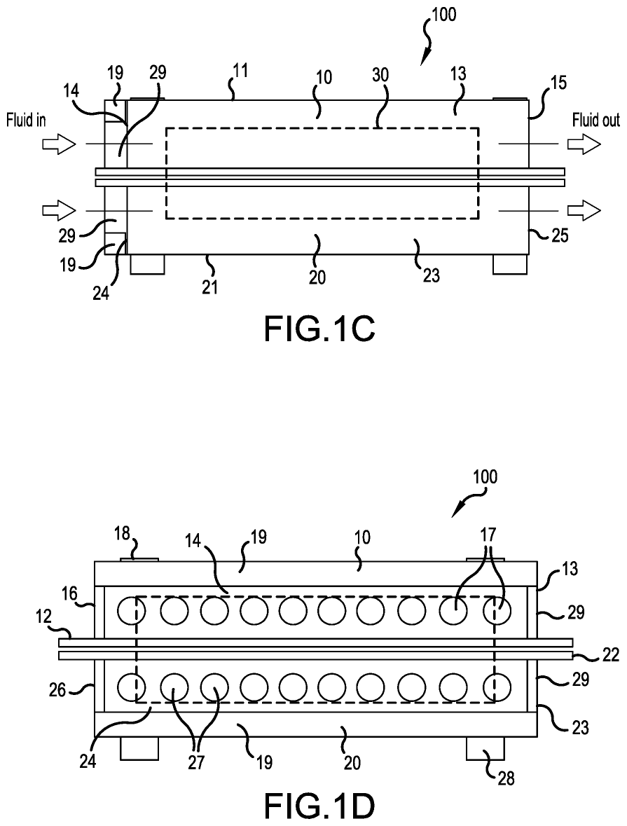

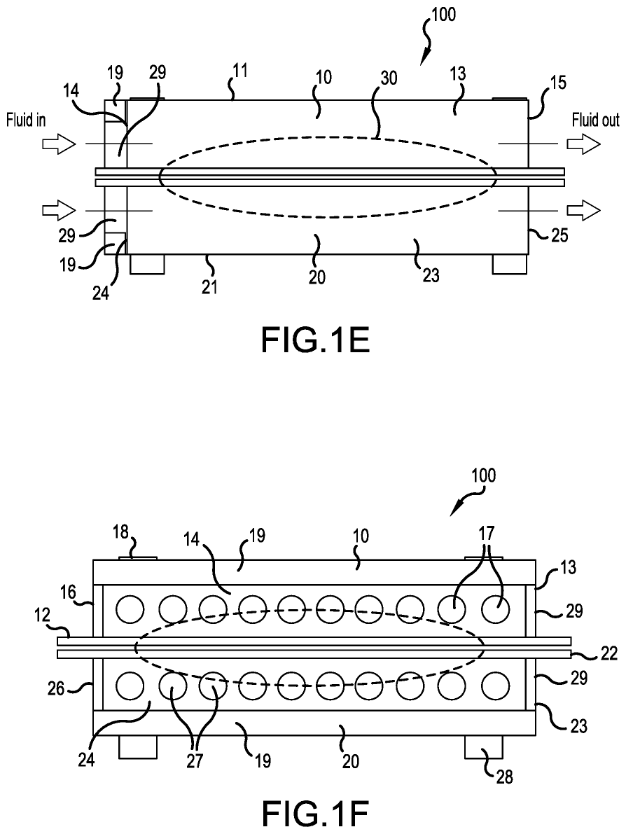

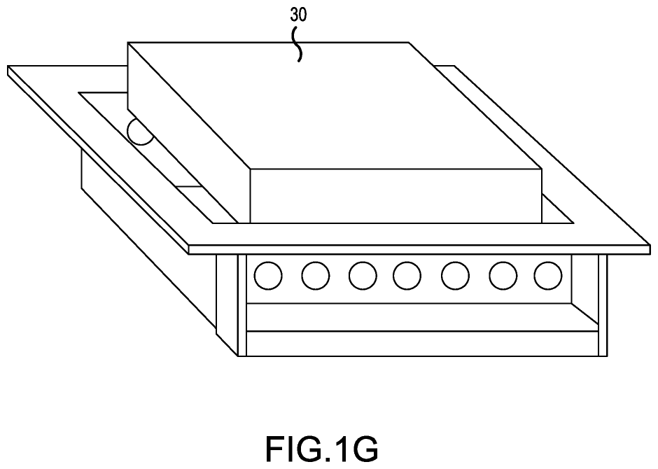

FIGS. 1A and 1B schematically illustrate side views of a secondary container 100 with flow-through apertures, according to an exemplary configuration provided herein. Secondary container 100 includes upper shell 10 and lower shell 20 and is configured to hold a primary container within an internal volume (primary container not specifically shown in FIGS. 1A-1B). Upper shell 10 includes upper surface 11, first flange 12, and a first plurality of sidewalls 13, 14, 15, 16 extending between the upper surface 11 and the first flange 12. Apertures 17 can be defined through first and second sidewalls of the first plurality of sidewalls. For example, in the configurations shown in FIGS. 1A-1B, apertures 17 extend through sidewall 14 at the “fluid in” side indicated with hollow arrows in FIG. 1A, and sidewall 15 at the “fluid out” side indicated with hollow arrows in FIG. 1A. Lower shell 20 of secondary container 100 includes lower surface 21, second flange 22, and a second plurality of sidewalls 23, 24, 25, 26 extending between the lower surface 21 and the second flange 22. Apertures 27 can be defined through first and second sidewalls of the second plurality of sidewalls. For example, in the configurations shown in FIGS. 1A-1B, apertures 27 extend through sidewalls 24 and 25. In some configurations, sidewalls 14 and 15 can be disposed parallel to one another. In some configurations, sidewalls 24 and 25 can be disposed parallel to one another and parallel to sidewalls 14 and 15. Optionally, in some configurations, sidewalls 13 and 16 can be disposed parallel to one another, and perpendicular to sidewalls 14 and 15. In some configurations, sidewalls 23 and 26 can be disposed parallel to one another and parallel to sidewalls 13 and 16, and perpendicular to sidewalls 24 and 25. Other configurations suitably can be used. It should be noted with reference to FIGS. 1A-1B, as well as the other figures herein, that the designation of shells or surfaces as “upper” or “lower” is merely a labeling convention, and not limiting of the various orientations in which the present secondary containers and shells suitably can be used.

First and second flanges 12, 22 contact one another to define an internal volume of secondary container 100. The flanges 12, 22 are securely engaged with one another using fasteners. Exemplary fasteners include, but are not limited to, quarter turn fasteners, spring-loaded fasteners, bolts with locking nuts, bolts with winged nuts, clamps, screws, rivets, pins, wire loops, snap-in fasteners, and polymer inserts.

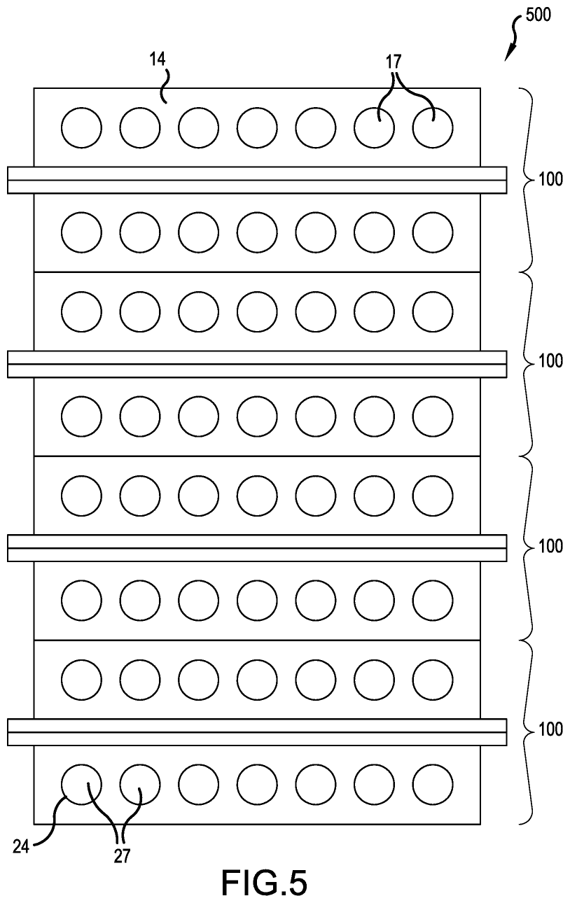

Upper and lower shells 10, 20 can have any suitable cross-sectional shape, e.g., can be generally rectangular or generally square. Upper surface 11 and lower surface 21 each can have any suitable shape, e.g., can be planar or substantially planar. Optionally, lower surface 21 includes legs 28, and upper surface 11 includes recesses 18 configured to receive and engage with the legs 28 of another container 100 so as to facilitate stacking of a plurality of containers 100 in a manner such as illustrated in FIG. 5. Sidewalls 13, 14, 15, 16 and sidewalls 23, 24, 25, 26 each can have any suitable shape, e.g., can be planar or substantially planar. Sidewalls 13, 14, 15, 16 can have any suitable angle(s) relative to upper surface 11, and sidewalls 23, 24, 25, 26 can have any suitable angle(s) relative to lower surface 21. For example, each of the sidewalls can have an angle of about 10-170° relative to the respective upper surface 11 or lower surface 21, e.g., an angle of about 30-150°, or an angle of about 50-130°, or an angle of about 70-110°, or an angle of about 80-100°, or an angle of about 90°.

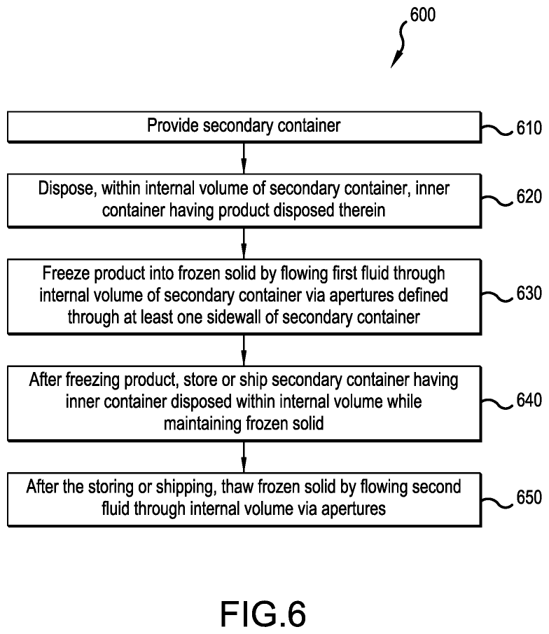

Apertures 17 defined through the first and second sidewalls (e.g., 14 and 15) of the first plurality of sidewalls 13, 14, 15, 16 and apertures 27 defined through the first and second sidewalls (e.g., 24 and 25) of the second plurality of sidewalls 23, 24, 25, 26 are configured such that fluid flows through the internal volume through those apertures. For example, apertures 17 and 27 can be located within respective sidewalls 14, 24 that face towards a source of fluid flow, such as but not limited to a fan or pump, system duct or system diffuser, orifice of pressurized vessel, wind blowing, or other suitable mechanism that causes flow of fluid in a direction shown in hollow arrows in FIG. 1A. In one nonlimiting example, the fluid is a gas, such as air, nitrogen, or argon. However, it should be appreciated that container 100 and the methods provided herein suitably can be used with fluids that are liquid. The fluid flow around and through container 100 can cause areas of high static pressure, e.g., at sidewalls 14, 24, as well as areas of low static pressure, e.g., at sidewalls 15, 25 which face away from the source of fluid flow. The pressure difference pressure between the high static pressure areas and the low static pressure areas causes flow of the fluid through the internal volume of secondary container 100. Fluid flowing through the internal volume of secondary container 100 flows around, past, and in contact with the inner container disposed therein, and thus can transfer heat to (as in thawing) or from (as in freezing) a product within the inner container, e.g., such as described in greater detail below with reference to FIG. 6.

Apertures 17 and 27 can have any suitable shape(s), such as slots, squares, rectangles, circles, ovals, or triangles, and any suitable size(s) and spacing(s). The apertures can be shaped, sized, and/or spaced the same as one another. Alternatively, one or more of the apertures can be shaped and/or sized and/or spaced differently than one or more other of the apertures. For example, the apertures can be shaped, sized, and spaced the same as one another on the fluid entry and exit sides, or they can differ. For example, the apertures can include horizontal slots on the entry side and vertical slots on the exit side, or vice versa. Or, for example, the apertures on the entry side can be larger or smaller than the apertures on the exit side. In one nonlimiting example, apertures 17, 27 are circular, have a diameter of about 0.8 inches (about 20.32 millimeters), and a spacing of about 1.25 inches (about 31.75 millimeters) (center-to-center). The apertures 17, 27 can be of other suitable shape or shapes, such as but not limited to square, rectangular, triangular, or oval. The orifices open area can provide sufficient flow rate as well as flow vorticity inside the secondary container to enhance heat transfer. Bridges (remaining material) between orifices can provide sufficient structural strength of the secondary container, e.g., for stacking. The examples of orifice size can, for example, be in the range of 0.1 inch (2.54 millimeters) to 16 inches (40.64 centimeters) depending on container size and can be extended to the range of 0.01 (0.254 millimeters) to 36 inches (91.44 centimeters), or other suitable size.

Optionally, one or both of the upper and lower shells 10, 20 can include one or more structural members configured to build pressure, e.g., to increase static pressure in area(s) of high static pressure and/or to increase a rate of flow of the fluid through the internal volume, such as at sidewalls 14 and 24 at the “fluid in” side indicated with hollow arrows in FIG. 1A. For example, the pressure building member of the upper and/or lower shell 10, 20 can include a linear bar 19 disposed outside of the internal volume along the apertures 17, 27 defined through the corresponding sidewall (e.g., sidewall 14 and/or 24). Additionally, or alternatively, the pressure building member of the upper and/or lower shell 10, 20 can include a fence 29 disposed outside of the internal volume around the apertures defined through the first sidewall of the first plurality of sidewalls disposed outside of the internal volume along the apertures 17, 27 defined through the corresponding sidewall (e.g., sidewall 14 and/or 24).

The pressure building member(s) can have any suitable structure and can be attached to container 100 in any suitable location. For example, the pressure building member(s) can be or include one or more bars, which can have any suitable cross-sectional shape such as rectangular, square, semi-spherical, semi-elliptical, triangular, or prismatic, or in the form of a plate. Additionally, or alternatively, the pressure building member(s) can include side plates (fences) configured so as to block, inhibit, or eliminate gas flow around the outside of container 100 (as opposed to through container 100). Additionally, or alternatively, the pressure building member(s) can be attached to the sidewall(s) having the apertures defined therethrough, can be attached to the sidewalls adjacent to the sidewall(s) having the apertures defined therethrough, and/or can be attached to the upper or lower surface at a region adjacent to the sidewall(s) having the apertures defined therethrough. In some configurations, the pressure building member(s) can be integrally formed with container 100. In other configurations, the pressure building member(s) can be formed separately from container 100 and attached using adhesive or a suitable fastener. In one nonlimiting example, the pressure building member can be detachable and replaced with a member of different geometry.

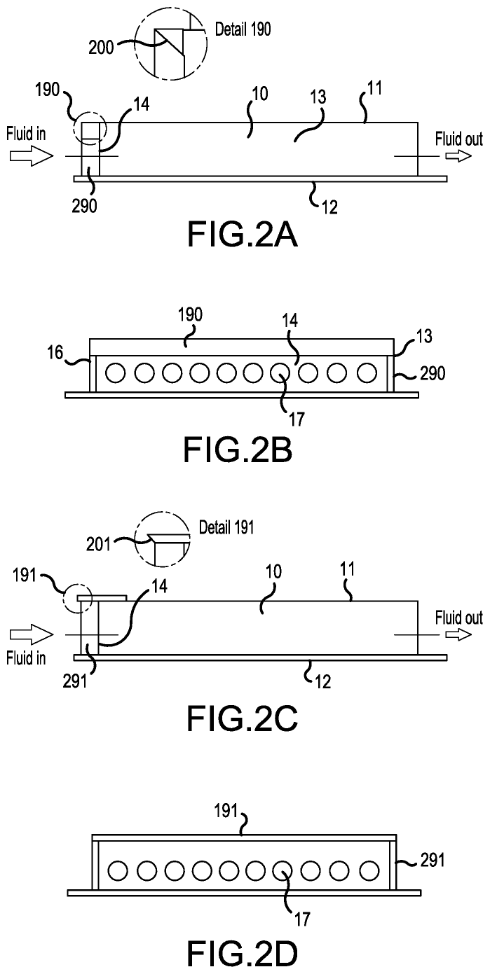

FIGS. 2A-2B schematically illustrate side views of an upper part (shell) 10 of the secondary container, according to an exemplary configuration provided herein. In FIGS. 2A-2B, pressure building members 190 and 290 can be coupled to the sidewall 14 facing the source of fluid flow. Pressure building member 190 can be configured as a bar coupled to sidewall 14 above apertures 17, adjacent to upper surface 11. Pressure building member 190 can have any suitable cross-section configured to build static pressure at sidewall 14 by reducing or inhibiting flow of the fluid onto and across upper surface 11, e.g., the exemplary cross section illustrated in the inset of FIG. 2A. In this exemplary configuration, pressure building member 190 includes a triangular bar with angled face 200 that directs fluid flow downward and away from upper surface 11. In FIGS. 2A-2B, pressure building members 290 can be configured as side plates coupled to sidewall 14 adjacent to sidewalls 13, 16 and configured to build static pressure at sidewall 14 by reducing or inhibiting flow of the fluid onto and across sidewalls 13 and 16. These side plates 290 can work in combination with pressure building members 200 to provide cavities in front of the secondary container.

FIGS. 2C-2D schematically illustrate side views of an upper part (shell) 10 of the secondary container, according to another exemplary configuration provided herein. In FIGS. 2C-2D, pressure building members 191 and 291 can be coupled to the upper face 11 and sidewalls 13, 16, respectively and face the source of fluid flow. Pressure building member 191 can be configured as a plate coupled to upper surface 11 above apertures 17, adjacent to sidewall 14. Pressure building member 191 can have any suitable cross-section configured to build static pressure at sidewall 14 by reducing or inhibiting flow of the fluid onto and across upper surface 11, e.g., the exemplary cross section illustrated in the inset of FIG. 2C. In this exemplary configuration, pressure building member 191 includes a plate with angled face 201 that directs fluid flow downward and away from upper surface 11. In FIGS. 2C-2D, pressure building members 291 can be configured as side plates coupled to sidewall 14 adjacent to sidewalls 13, 16 and configured to build static pressure at sidewall 14 by reducing or inhibiting flow of the fluid onto and across sidewalls 13 and 16. These side plates 291 can work in combination with pressure building members 201 to provide cavities in front of the secondary container.

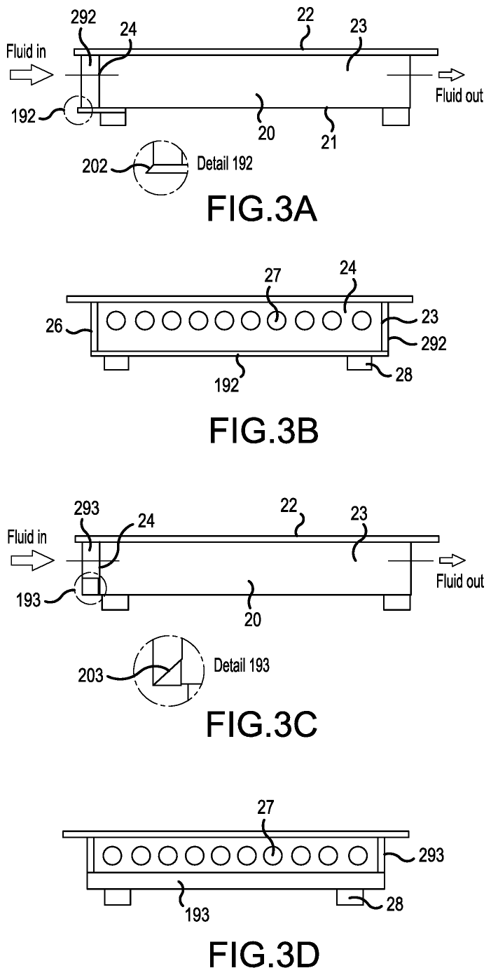

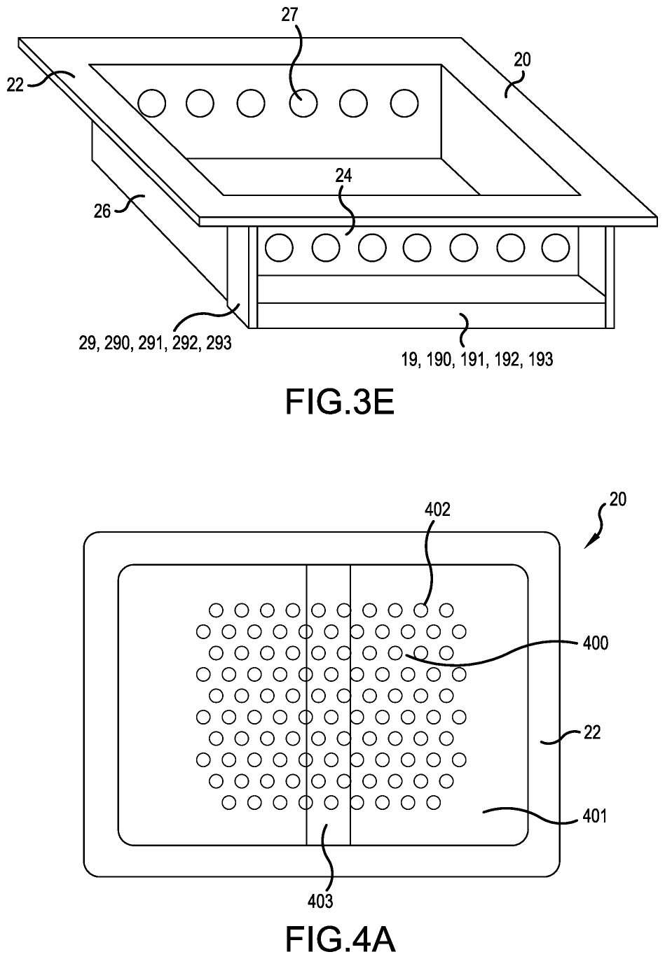

FIGS. 3A-3B schematically illustrate side views of a lower part (shell) 20 of the secondary container, according to an exemplary configuration provided herein. In FIGS. 3A-3B, pressure building members 192 and 292 can be coupled to the sidewall 24 facing the source of fluid flow. Pressure building member 192 can be configured as a bar coupled to lower surface 21 below apertures 27. Pressure building member 192 can have any suitable cross-section configured to build static pressure at sidewall 24 by reducing or inhibiting flow of the fluid onto and across lower surface 21, e.g., the exemplary cross section illustrated in the inset of FIG. 3A. In this exemplary configuration, pressure building member 192 includes a triangular bar with angled face 202 that directs fluid flow upward and away from upper surface 11. In FIGS. 3A-3B, pressure building members 292 can be configured as side plates coupled to sidewall 24 adjacent to sidewalls 23, 26 and configured to build static pressure at sidewall 24 by reducing or inhibiting flow of the fluid onto and across sidewalls 23 and 26. These side plates 292 can work in combination with pressure building members 202 to provide cavities in front of the secondary container.

FIGS. 3C-3D schematically illustrate side views of a lower part (shell) 20 of the secondary container, according to another exemplary configuration provided herein. In FIGS. 3C-3D, pressure building members 193 and 293 can be coupled to the sidewall 24 facing the source of fluid flow. Pressure building member 193 can be configured as a plate coupled to lower surface 21 below apertures 27, adjacent to sidewall 24. Pressure building member 193 can have any suitable cross-section configured to build static pressure at sidewall 24 by reducing or inhibiting flow of the fluid onto and across lower surface 21, e.g., the exemplary cross section illustrated in the inset of FIG. 3C. In this exemplary configuration, pressure building member 193 includes a plate with angled face 203 that directs fluid flow upward and away from lower surface 21. In FIGS. 3C-3D, pressure building members 293 can be configured as side plates coupled to sidewall 24 adjacent to sidewalls 23, 26 and configured to build static pressure at sidewall 24 by reducing or inhibiting flow of the fluid onto and across sidewalls 23 and 26. These side plates 293 can work in combination with pressure building members 203 to provide cavities in front of the secondary container.

FIG. 3E schematically illustrates a perspective view of a lower part of the secondary container (shell) 20, according to an exemplary configuration provided herein. Exemplary locations of pressure building members 190, 191, 192, 193 and 290, 291, 292, 293 are illustrated in FIG. 3E. In FIGS. 2A-3E, pressure building members 190, 191, 192, 193 and 290, 291, 292, 293 can be integrally formed with lower shell 20, or can be formed separately and attached using adhesive or a suitable fastener. In one nonlimiting example, the upper surface, the first plurality of sidewalls, and the first flange of the upper shell 10 are of unitary construction; the lower surface, the second plurality of sidewalls, and the second flange of the lower shell 20 are of unitary construction; and the first and second flanges are separable from one another.

Referring again to FIGS. 1A-1B, a primary container can be disposed within the internal volume of the secondary container 100 in any suitable manner. For example, FIGS. 1C-1D schematically illustrate side views of a secondary container holding an example primary container 30, according to an exemplary configuration provided herein. In the nonlimiting example illustrated in FIGS. 1C-1D, secondary container 100 encloses primary container 30 such as a prismatic product packaging, e.g., a box. FIGS. 1E-1F schematically illustrate side views of a secondary container holding another example primary container 30, according to an exemplary configuration provided herein. In the nonlimiting example illustrated in FIGS. 1E-1F, secondary container 100 encloses primary container 30 such as a bag. FIG. 1G schematically illustrates an exploded view of the secondary container of FIGS. 1C-1D having an exemplary primary container 30 disposed therein, according to an exemplary configuration provided herein. Note that in FIGS. 1C-1G, primary container 30 optionally can contain further packaging therein. As such, primary container 30 can be considered to be a “secondary” container, and secondary container 100 can be considered to be a “tertiary” container.

In configurations such as exemplified by FIGS. 1A-1G, 2A-2D, 3A-3E, the fluid (such as a gas) flows through the internal volume at a rate and/or at a temperature sufficient to cool or heat a product stored within the primary container 30 at an appropriate rate, e.g., so as to maintain quality and inhibit degradation of the product, which can be perishable. Illustratively, the product can include a frozen solid. The fluid can be at a temperature selected to thaw the frozen solid. As another example, the product can include a liquid. The fluid can be at a temperature selected to freeze the liquid. In one example, secondary container 100 is configured such that the fluid flows through the internal volume of the secondary container 100, and thus across primary container 30, at a rate of at least about 1 meter per second, or at least about 2 meters per second, or at least about 3 meters per second, or at least about 4 meters per second, or at least about 5 meters per second, or at least about 6 meters per second, or at least about 7 meters per second, or at least about 8 meters per second. Other exemplary rates of fluid flow include, but are not limited to, at least 0.001 meters per second, or at least 0.005 meters per second, or at least 0.05 meters per second, or at least 0.1 meters per second. For example, pressure building members 19, 190, 191, 192, or 193 and/or 29, 290, 291, 292, or 293 can be configured so as to build sufficient pressure within the internal volume of secondary container 100 to achieve flow rates such as exemplified herein. In some configurations, the fluid flows through a portion of the internal volume within the upper shell at a rate that is about the same as through a portion of the internal volume within the lower shell. However, it should be appreciated that flow rates through the upper and lower shells can be different than one another.

Primary container 30 can be supported within secondary container 100 using any suitable support member(s), such as flat or concave sheets (fabric, film, membrane). Such support member(s) optionally can include openings for better contact of fluid and the primary container. The openings (perforations) can be of various shapes, such as slots, squares, rectangles, circles, ovals, or triangles, and any suitable sizes. The openings can be shaped and sized the same as one another. Alternatively, one or more of the openings can be shaped and/or sized differently than one or more other of the openings. Additionally, or alternatively, the openings can be arranged symmetrically, or can be arranged asymmetrically. In some configurations, the support member(s) can be elastic.

For example, to increase heat transfer between the fluid and the wall of the primary (inner container), a membrane (support member) can be perforated such that a predefined percentage of the surface area of the primary container can be directly exposed to the fluid, without any insulating effect of the membrane. The perforation (opening) open area and holes pattern in the membrane can be selected so as to provide enhanced heat transfer in comparison to a solid (non-perforated) membrane while maintaining sufficient strength of the membrane to support the primary container. The membrane can be made of an elastomeric material such as, but not limited to, natural or synthetic rubber, elastomeric polymer, or stretchable fabric; therefore, after removal of some material to create perforations, the remaining bridges of membrane may stretch more than in a solid membrane. Such weakening of the membrane can be mitigated in any of a variety of ways. For example, the perforation pattern can be selected such that there are opening-free bands left in the membrane, what either follow the main stress directions of the membrane or are positioned in regions where the membrane material can stretch the least, or both. Additionally, or alternatively, the perforation pattern may be such that the membrane material bridges between the openings (perforations) can be strengthened by the use of additional structural members, such as straps (harnesses) using the membrane material, or another material, such as a similar elastomeric material such as, for example, natural or synthetic rubber, elastomeric polymer, or stretchable fabric, that can be attached to the membrane or to the flange of the respective upper or lower shell. Additionally, or alternatively, the perforation of the membrane can have a relatively high open area percentage, but the parts or the whole membrane can be supported by an elastomeric net which may include a material with an elastic characteristic similar to or stronger than that of the membrane material such as, but not limited to, natural or synthetic rubber, elastomeric polymer, or stretchable fabric. In such a manner, the assembly can have a relatively large open area (good heat transfer) and be sufficiently strong as not to sag under the weight of the primary container, e.g., due to the supporting effect of the elastomeric net. The elastomeric net can have a relatively large open area, and thus can be expected not to significantly affect contact between the fluid and the primary container. The material selection can include, but is not limited to, natural or synthetic rubber, elastomeric polymer, stretchable fabric, or coiled metal spring wires. The open area can be such to provide fluid contact with the primary container, yet substantially not to affect the primary container by concentration of stress. Elastic properties and ability of stretching can work over a broad range of temperatures, for example between −200° C. to +300° C., −200° C. to +150° C. or −200° C. to +100° C.

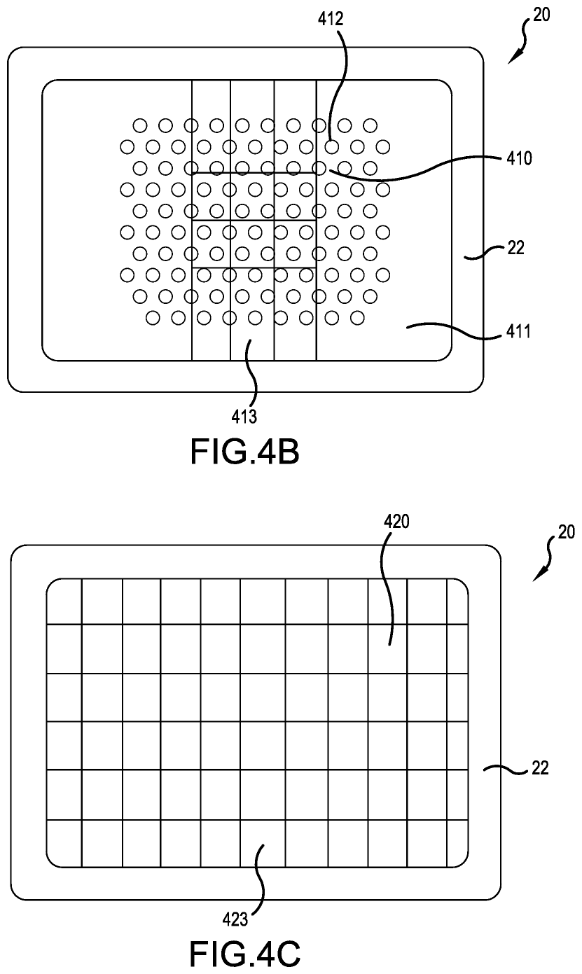

For example, FIGS. 4A-4C schematically illustrate plan views of a lower part (shell) 20 of the secondary container including exemplary support members for the primary (inner) container, according to exemplary configurations provided herein. In the nonlimiting example illustrated in FIG. 4A, lower shell 20 includes flange 22 and support member 400. Support member 400 includes membrane 401 having a plurality of openings 402 defined therethrough via which fluid passing through the interior volume can contact the primary container, and strap 403 configured to provide additional support to the primary container relative to membrane 401, which also supports the primary container. In the nonlimiting example illustrated in FIG. 4B, lower shell 20 includes flange 22 and support member 410. Support member 410 includes membrane 411 having a plurality of openings 412 defined therethrough via which fluid passing through the interior volume can contact the primary container, and elastic net or mesh 413 configured to provide additional support to the primary container relative to membrane 411, which also supports the primary container. Note that in FIGS. 4A and 4B, openings 402, 412 can have any suitable distribution, e.g., can be substantially evenly distributed across membrane 401, 411, or can be unevenly distributed across membrane 401, 411, e.g., can be distributed in a central portion of the respective membrane and not distributed in a periphery of that membrane. In the nonlimiting example illustrated in FIG. 4C, lower shell 20 includes flange 22 and support member 420. Support member 420 includes elastic net or mesh 424 configured to support the primary container and has an open weave to allow fluid passing through the interior volume to contact the primary container. In other configurations, support members can include fabrics. For example, any of membranes 401, 411, elastic net or mesh 413, and elastic net or mesh 424 suitably can be replaced with a fabric of sufficient strength to support the primary container and of sufficient porosity to facilitate contact between the fluid and the primary container.

It should be appreciated that the upper shell 10 can include a support member configured similarly as any of the support members illustrated with respect to lower shell 20. For example, the upper shell can include a first support member and the lower shell can include a second support member, and the first and second support members can be configured to support an inner container within the internal volume. The support members of the upper and lower shells can be the same as one another, or can be different than one another. For example, support members independently can be selected from the group consisting of a membrane, a fabric, a strap, a net, a sheet, a film, a foil, a mesh, cables, bands, ribbons, cords, bars, rods, and screens, and any suitable combinations thereof. Optionally, the first and second support members each can be elastic. In one example, the first and/or second support members are pre-shaped to accommodate the inner container, for example as two halves of a mesh basket holding the inner container within the internal volume. Additionally, or alternatively, the first and second support members each can include perforations defined therethrough, and the fluid can contact the primary container via the perforations. In one example, the support members of the upper and lower shells each can include a membrane, and the membranes of the upper and lower shells can have detachable flanges which stay attached together. After freezing and transport of the assembly to a thawing area (e.g., in a manner such as described with reference to FIG. 6), the two membranes with their flanges can be removed from the container (flanges stay closed and membranes hold the frozen primary container). Then the resulting assembly can be thawed in the open, e.g., on a tray, or placed into another container (such as a bag) and placed in a thawing liquid bath for fast thawing. The assembly alternatively can be placed into a thawing machine, e.g., that uses warming bladders or pads. Usefully, the frozen primary container is not handled directly, so as to inhibit damage to that container. After thawing, the possibility of such damage is reduced.

The present secondary containers suitably can be used to freeze, thaw, and transport products that are held within a wide variety of shapes, types, and sizes of primary (inner) containers. Examples of primary containers can include, but are not limited to, boxes, bags, vials, and/or bottles. The primary containers optionally can be placed in secondary containers, for example, the vials placed in boxes and those boxes can be then placed in the presently provided containers which then can be considered tertiary containers. Additionally, the secondary (outer) containers provided herein can be provided in the form of a kit suitable for assembly at a desired location. Such a kit can, for example, include an upper shell and a lower shell. The upper shell can be configured similarly as discussed elsewhere herein, e.g., can include an upper surface, a first flange, and a first plurality of sidewalls extending between the upper surface and the first flange, wherein apertures are defined through first and second sidewalls of the first plurality of sidewalls. The lower shell can be configured similarly as discussed herein, e.g., can include a lower surface; a second flange, a second plurality of sidewalls extending between the lower surface and the second flange, wherein apertures are defined through first and second sidewalls of the second plurality of sidewalls. The first and second flanges can be configured to be brought into contact with one another to define an internal volume.

Additionally, the present secondary containers can be readily stackable, and configured so as to facilitate freezing, thawing, and/or transport of a product, all while stacked. For example, FIG. 5 schematically illustrates a side view of an assembly including a stack 500 of secondary containers with flow-through apertures, according to exemplary configurations provided herein. In exemplary stack 500 illustrated in FIG. 5, five assembled secondary (outer) containers 100 are stacked, although it should be appreciated that any suitable number of containers 100 can be stacked. The stacked containers 100 each can be arranged such that the sidewalls within which apertures 17 and 27 are located (e.g., sidewalls 14, 24, respectively) face towards a source of fluid flow, thus facilitating substantially simultaneous flow of fluid through each of the containers, and therefore facilitating substantially simultaneous freezing or thawing of products within such containers. Optionally, each of the stacked containers 100 includes pressure building member(s), e.g., such as described with reference to FIGS. 1A-1G, 2A-2D, and 3A-3E. Additionally, or alternatively, each of the stacked containers 100 optionally includes support member(s), e.g., such as described with reference to FIGS. 4A-4C.

It should be appreciated that the present secondary (outer) containers can be used in any suitable process for handling a primary container, e.g., for freezing, thawing, storing, and/or shipping product(s) stored within a primary container. For example, FIG. 6 illustrates a flow of operations in an exemplary method 600 for handling a primary container using a secondary container with flow-through apertures, according to exemplary configurations provided herein. Method 600 illustrated in FIG. 6 includes providing a secondary container (operation 610). The secondary container can be configured such as described elsewhere herein, e.g., container 100 illustrated in FIGS. 1A-1G.

Method 600 illustrated in FIG. 6 also can include disposing, within an internal volume of the secondary container, an inner container having a product disposed therein (operation 620). In one nonlimiting example, the inner container can be placed on the support structure(s) of the lower shell, and the upper shell then coupled to the lower shell, e.g., by securably contacting the flange of the upper shell with the flange of the lower shell. As noted above, in circumstances where the inner container contains additional packaging, that additional packaging can be considered a primary container, the inner container can be considered to be a secondary container, and the outer packaging provided herein can be considered to be a tertiary container.

Method 600 illustrated in FIG. 6 also can include freezing the product into a frozen solid (operation 630). In one example, operation 630 includes freezing the product into a frozen solid by flowing a first fluid through the internal volume via apertures defined through at least one sidewall of the secondary container, wherein the first fluid is at a temperature selected to freeze the product. In one example, the product can include a liquid that is frozen during operation 630.

Method 600 illustrated in FIG. 6 also can include, after freezing the product, storing or shipping the secondary container having the inner container disposed within the internal volume while maintaining the frozen solid (operation 640). For example, the product can be kept in the frozen solid state during any suitable combination of shipment and storage for any suitable period of time.

Method 600 illustrated in FIG. 6 also can include, after the storing or shipping, thawing the frozen solid by flowing a second fluid through the internal volume via the apertures, wherein the second fluid is at a temperature selected to thaw the frozen solid (operation 650). The first and second fluids can be the same as one another (e.g., can be air or other nonreactive gas), or can be different than one another. In one example, the product can include a liquid that is thawed back into a liquid during operation 650.

It should be appreciated that any suitable subcombination(s) of steps of FIG. 6 suitably can be implemented. For example, a liquid product can be frozen using operations that include disposing, within an internal volume of a secondary container, the inner container having the product disposed therein; and freezing the liquid by flowing fluid through the internal volume via apertures defined through at least one sidewall of the secondary container, wherein the fluid is at a temperature selected to freeze the liquid. Such a combination of steps may be implemented by a single party. As another example, a product that includes a frozen solid can be thawed using steps that include disposing, within an internal volume of a secondary container, the inner container having the product disposed therein; and thawing the frozen solid by flowing fluid through the internal volume via apertures defined through at least one sidewall of the secondary container, wherein the fluid is at a temperature selected to thaw the frozen solid. Such a combination of steps may be implemented by a single party. Other permutations of freezing, thawing, storing, and/or shipping products can be implemented.

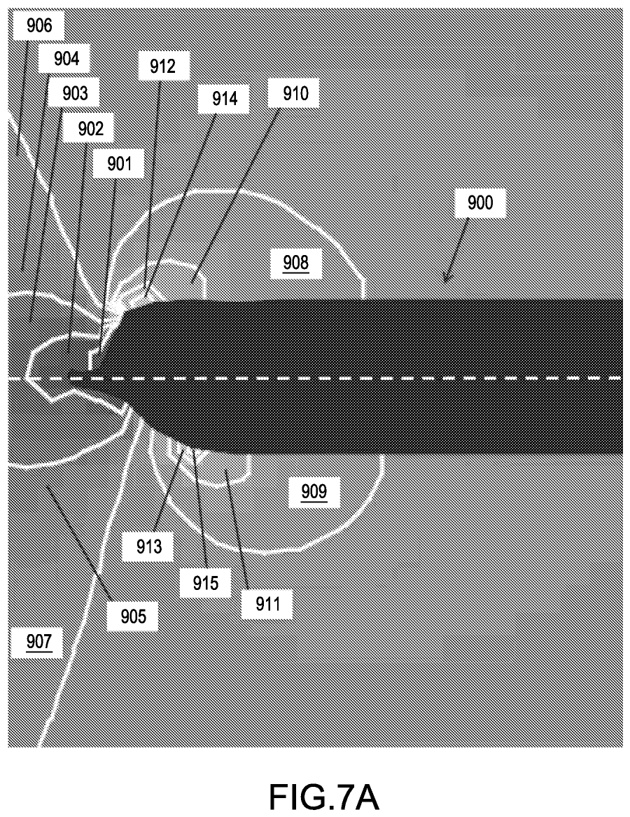

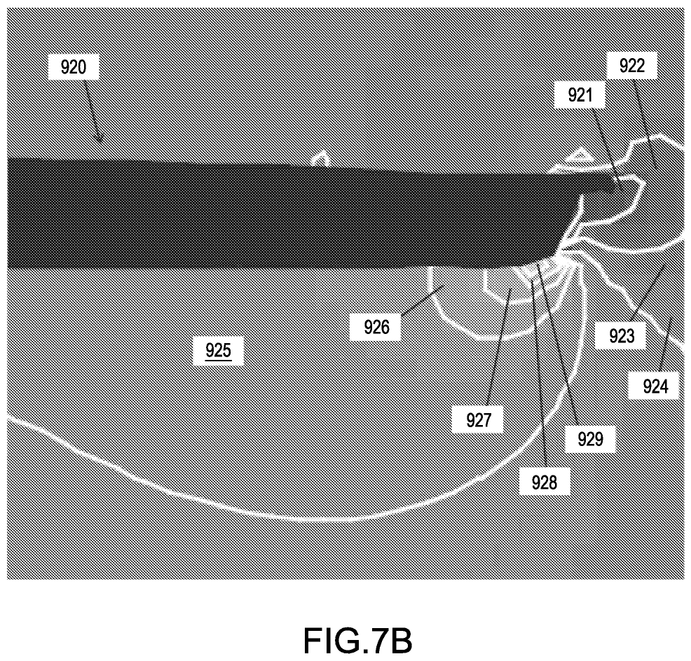

FIGS. 7A-7B schematically illustrate exemplary static pressure distributions around an exemplary secondary container 900 and an exemplary lower shell 920 of a secondary container with flow-through apertures, according to one nonlimiting example. For example, FIGS. 7A-7B show the static pressure distribution on the secondary container walls (the gas flow is from left to right). The blue colors or the zones identified by reference numbers 901, 902, 903, 904, and 905 indicate the positive static pressure. The red color or the zones identified by reference numbers 906, 907, 908, and 909 indicate negative static pressure. The orange color or the zones identified by reference numbers 910, 911, and 912 indicate higher negative static pressure. The yellow color or the zones identified by reference numbers 913, 914, and 915 indicate even higher negative static pressure. In FIG. 7A, static pressure distributions in a gas flow are shown around the secondary container model (black contour). In FIG. 7A, two modeled front sidewall angles are shown, divided by the white dotted line. The steeper sidewall angle (top model) shows higher static pressure than the lower sidewall angle (bottom model). A completely vertical sidewall would be expected to provide even higher static pressure (not shown due to modeling limitations). The positive pressure zone (blue color or the zones identified by reference numbers 901, 902, 903, 904, and 905) is accompanied by a highly negative pressure zone (yellow color or the zones identified by reference numbers 913, 914, and 915). The sidewall includes apertures in the zone of high positive static pressure (901, 902, 903, 904, and 905). Static pressure buildup is a driving force for the gas flow through the secondary container. FIG. 7B, static pressure distributions in a gas flow are shown around the lower shell 920 of secondary container model (black contour). The blue colors or the zones identified by reference numbers 921, 922, and 923 indicate the positive static pressure. The red color or the zones identified by reference numbers 924 and 925 indicate negative static pressure. The orange color or the zones identified by reference numbers 926, 927 and 928 indicate higher negative static pressure. The yellow color or the zones identified by reference number 929 indicate even higher negative static pressure. The primary container would be placed on top of the lower shell 920 of the secondary container. The primary container can be supported, for example, by a flat or concave sheet, or stretchable or elastic film. These support member(s) can be perforated to facilitate contact of the primary container with gas flowing through the inner volume of the secondary container. Such perforation can enhance heat transfer between the fluid and the primary container (e.g., in either cooling or heating).

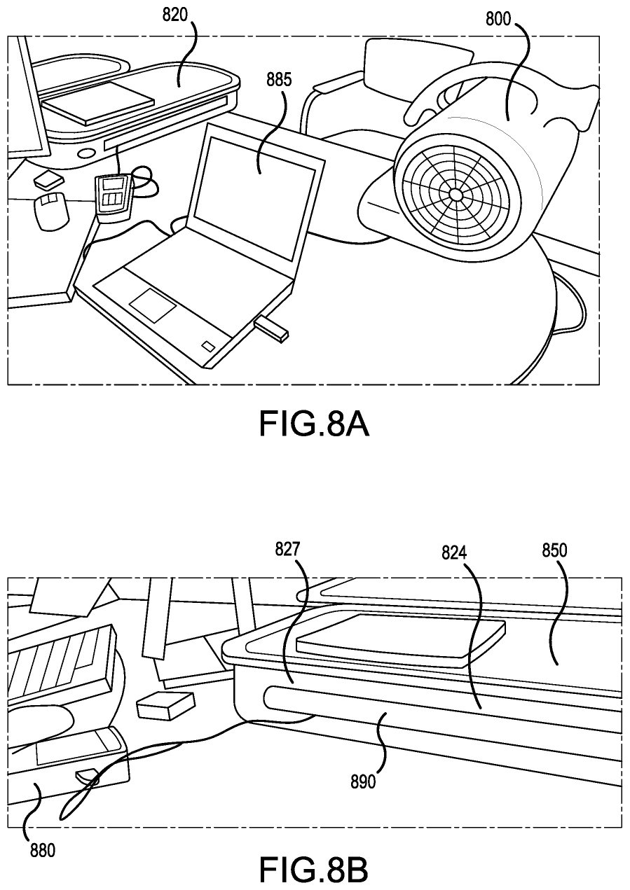

FIGS. 8A-8B are photographs of a demonstration of the effect of pressure building members, according to one nonlimiting example. The source of fluid flow was a blower 800 configured to provide a flat jet of air at high velocity. The blower 800 had three speeds, and was set to provide air velocities of about 4-6 meters/second in front of the secondary container, which included a lower shell 820 having front apertures 827 defined in front sidewall 824, rear apertures defined in a rear sidewall (rear apertures and rear sidewalls not shown in FIGS. 8A-8B), an elastomeric membrane 850 provided as a support structure, and a pipe 890 attached to sidewall 824 below apertures 827 and provided as a pressure building member. A notebook was used to cover apertures in the elastomeric membrane 850. Gas velocities were measured using anemometer 880 in front of lower shell 820, as well as inside of lower shell 820 by inserting the probe of the anemometer through one of apertures 827. The computer 885 was used to record velocities measured by the anemometer 880. Velocities inside of the container first were measured without the pressure building member (pipe) 890, and were in the range of about 0.4 to 0.7 meters/second. After attaching the pressure building member 890, the velocities were observed to increase to about 1 to 2 meters/second. Other velocities such as exemplified elsewhere herein suitably may be used.

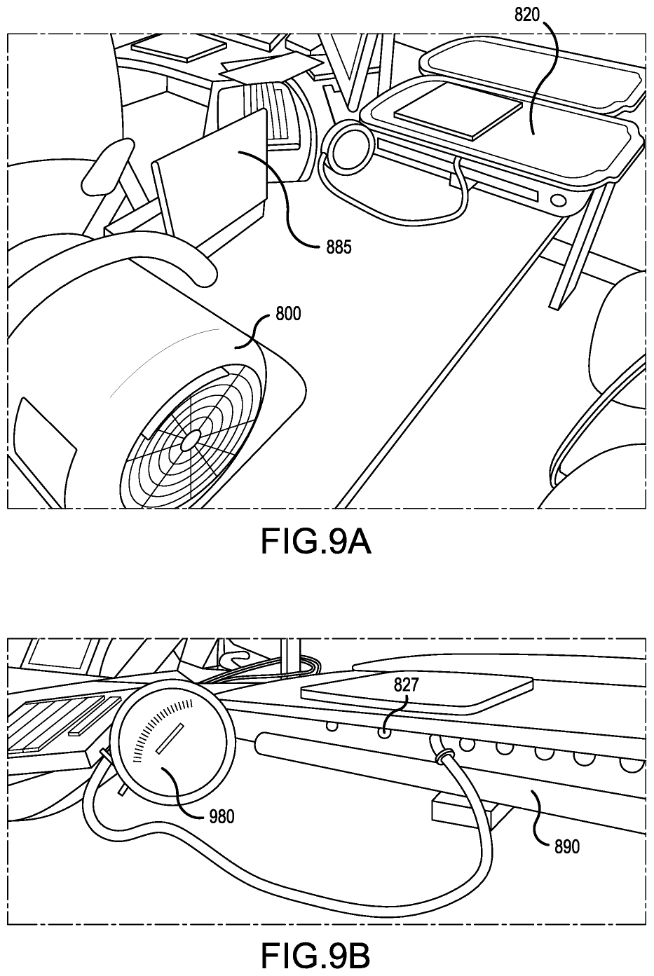

FIGS. 9A-9B are photographs of another demonstration of the effect of pressure building members, according to one nonlimiting example. In this demonstration, the pressure buildup in front of lower shell 820 above the pressure building member 890 was measured using a manometer 980 at the approximate level of apertures 827. Placing the manometer tube at different locations along pressure building member 890 showed a pressure buildup of about 0.15 (3.81 millimeter) to 0.2 inch (5.08 millimeter) water gauge (about 3.8 to 5.0 mm H₂O). This corresponds to the buildup of static pressure that causes a pressure differential between the front and back of the container, thus causing fluid flow through the container.

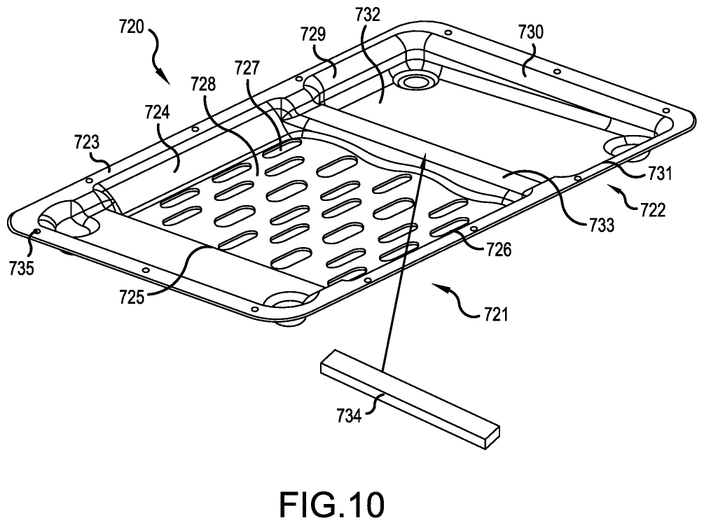

FIG. 10 schematically illustrates a perspective view of a lower shell 720 of another example of a secondary container 700 (not shown in FIG. 10, shown in assembled form only in FIG. 13) with flow-through apertures, according to an exemplary configuration provided herein. The secondary container 700, which is formed from an upper shell 710 (not shown in FIG. 10, shown in FIG. 13) and an identical lower shell 720, is configured to hold a primary container within an internal volume (primary container shown in FIG. 15). Lower shell 720 will be described in reference to FIGS. 10-12; however, it should be understood that upper shell 710 is identical to lower shell 720.

Referring to FIG. 10, lower shell 720 includes a first compartment 721, a second compartment 722, and a flange 723 surrounding the first compartment 721 and the second compartment 722. The first compartment 721 includes a plurality of walls, including a first wall 724, a second wall 725, and a third wall 726, and a floor 728. Apertures 727 can be defined through the floor 728 of the first compartment 721. Similarly, the second compartment 722 includes a plurality of walls, including a first wall 729, a second wall 730, and a third wall 731, and a floor 732. Between the first compartment 721 and the second compartment 722, a junction 733 may separate the two compartments 721, 722. The first compartment 721 may have a floor 728 with curvature and, in an example, the first compartment 721 may receive a bag of a primary container. The second compartment 722 may have a floor 732 with a flat surface and, in an example, the second compartment 722 may receive tubing of the primary container.

Still referring to FIG. 10, a pinch 734 may be placed on the junction 733 where the compartments 721, 722 are separated to inhibit the vertical movement of the primary container, such as vertical movement of a bag of the primary container, and protect the primary container, such as an interface of the tubing to the bag of the primary container. In an example, the pinch 734 may be formed of a foam material; however, any other material may be used such as an elastic material or any other material as would be known to a person having ordinary skill in the art. In a specific nonlimiting example, the pinch 734 may be an extruded silicone sponge having a rectangular block shape with dimensions of 8 inches (20.32 centimeters) by 1 inch (2.54 centimeters) by 0.5 inches (1.27 centimeters). However, pinch 734 can have any suitable shape, such square, rectangle, circle, oval, or triangle, and any suitable size. The pinch 734 may be attached to the junction 733 using any adhesive or may be integrally formed. In a specific nonlimiting example, the pinch 734 is attached to the junction 733 using an acrylic pressure sensitive adhesive.

The flange 723 may include one or more holes 735 for receiving a fastening element which may secure the lower shell 720 to an upper shell 710. In an example, twelve holes 735 are provided in the flange 723 although any number of one or more holes 735 may be used. It should be noted with reference to FIG. 10, as well as the other figures herein, that the designation of shells or surfaces as “upper” or “lower” is merely a labeling convention, and not limiting of the various orientations in which the secondary containers and shells suitably may be used.

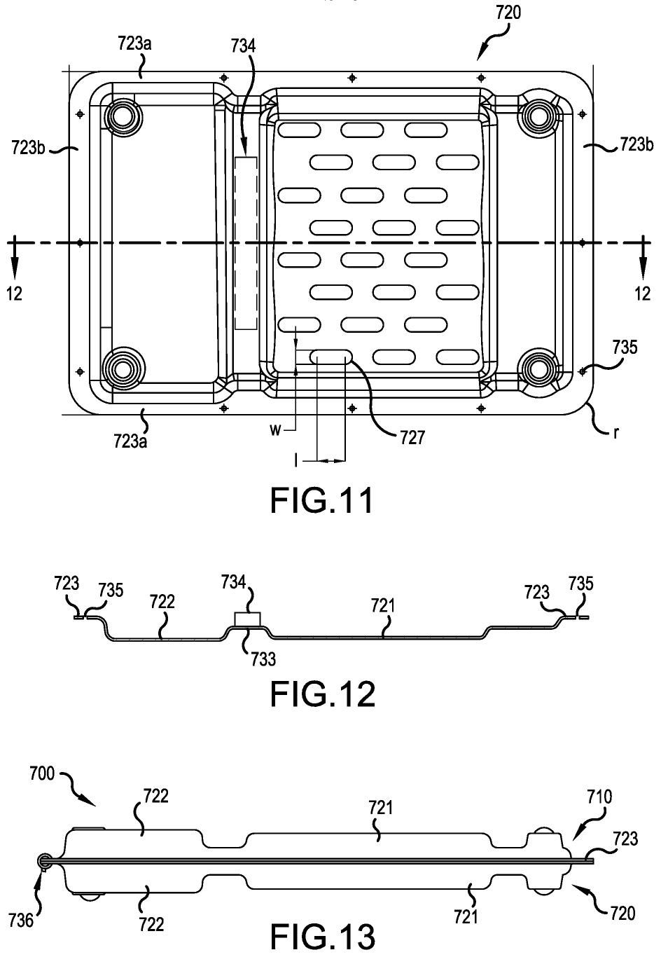

FIG. 11 schematically illustrates a top planar view of the lower shell 720 with flow-through apertures, according to an exemplary configuration provided herein.

Referring to FIG. 11, the lower shell 720 and the pinch 734 are illustrated. The flange of the lower shell 723 extends all around the lower shell 720 and includes two pairs of parallel flange sides 723a, 723b. The first pair of flange sides 723a may be longer than the second pair of flange sides 723b so that the lower shell 720 has a generally rectangular shape. However, lower shell 720 can have any suitable cross-sectional shape, e.g., can be generally rectangular or generally square, among other shapes. For example, the first pair of flange sides 723a may measure approximately 20-30 inches (approximately 50.8-76.2 centimeters) and the second pair of flange sides may measure approximately 10-20 inches (approximately 25.4-50.8 centimeters). In a specific nonlimiting example, the first pair of flange sides 723a may measure approximately 24.4 inches (approximately 619.76 millimeters) and the second pair of flange sides may measure approximately 16.0 inches (approximately 40.64 centimeters). The lower shell 720 may have rounded corners where each of the first pair of flange sides 723a meets each of the second pair of flange sides 723b. The radius of curvature r of each of the corners may measure, for example, approximately 0.5 to 2.5 inches (approximately 1.27 to 6.35 centimeters). In a specific nonlimiting example, the radius of curvature r measures 1.5 inches (3.81 centimeters).

The apertures 727 of the lower shell are also illustrated in FIG. 11. Apertures 727 can have any suitable shape(s), such as slots, squares, rectangles, circles, ovals, or triangles, and any suitable size(s) and spacing(s). The apertures 727 can be shaped, sized, and/or spaced the same as one another. Alternatively, one or more of the apertures 727 can be shaped and/or sized and/or spaced differently than one or more other of the apertures 727. For example, the apertures 727 can be shaped, sized, and spaced the same as one another on the upper shell 710 and lower shell 720, or they can differ. For example, the apertures 727 can include horizontal slots on the upper shell 710 and vertical slots on the lower shell 720, or vice versa. Or, for example, the apertures 727 on the upper shell 710 can be larger or smaller than the apertures 727 on the lower shell 720. For example, the apertures 727 may have a length of about 0.2-3 inches (about 5.08-76.2 millimeters) and a width of about 0.5-3.5 inches (about 1.27-8.89 centimeters). In a specific nonlimiting example, apertures 727 are oval, have a length of about 1.3 inches (about 33.02 millimeters), and a width of about 0.7 inches (about 17.78 millimeters). The apertures 727 can be of other suitable shape or shapes, such as but not limited to square, rectangular, triangular, or circular.

Still referring to FIG. 11, the holes 735 of the flange 723 may also have any suitable shape(s), such as slots, squares, rectangles, circles, ovals, or triangles, and any suitable size(s) and spacing(s). The holes 735 can be shaped, sized, and/or spaced the same as one another. Alternatively, one or more of the holes 735 can be shaped and/or sized and/or spaced differently than one or more other of the holes 735. For example, the holes 735 can be shaped, sized, and spaced the same as one another on the upper shell 710 and lower shell 720, or they can differ. For example, the holes 735 on the upper shell 710 can be larger or smaller than the holes 735 on the lower shell 720. For example, the holes 735 may have a diameter of about 0.05-0.9 inches (about 1.27-22.86 millimeters). In a specific nonlimiting example, the holes 735 are circular and have a diameter of about 0.2 inches (5.08 millimeters). The holes 735 can be of other suitable shape or shapes, such as but not limited to square, rectangular, triangular, or oval.

FIG. 12 schematically illustrates a cross-sectional view of the lower shell 720 at the line 12-12 of FIG. 11, according to an exemplary configuration provided herein.

Referring to FIG. 12, the lower shell 720 and the pinch 734 are illustrated. The pinch 734 is separating the first compartment 721 from the second compartment 722 and is positioned above juncture 733. In this cross-section, two holes 735 of the flange 723 are illustrated.

FIG. 13 schematically illustrates a side view of the secondary container 700 including the upper shell 710 and the lower shell 720, according to an exemplary configuration provided herein. Referring to FIG. 13, the flanges 723 of the upper shell 710 and the lower shell 720 contact one another to define an internal volume of secondary container 700. As a result, two internal volumes are formed by the first compartment 721 and the second compartment 722. The flanges 723 are securely engaged with one another using a fastener 736. Exemplary fasteners 736 include, but are not limited to, quarter turn fasteners, spring-loaded fasteners, bolts with locking nuts, bolts with winged nuts, clamps, screws, rivets, pins, wire loops, snap-in fasteners, and polymer inserts. In a specific nonlimiting example, the fastener 736 is a high performance cable tie such as a 7″ nylon cable tie. However, it should be appreciated that any type, size, or material fastener may be used as is known by one having ordinary skill in the art.

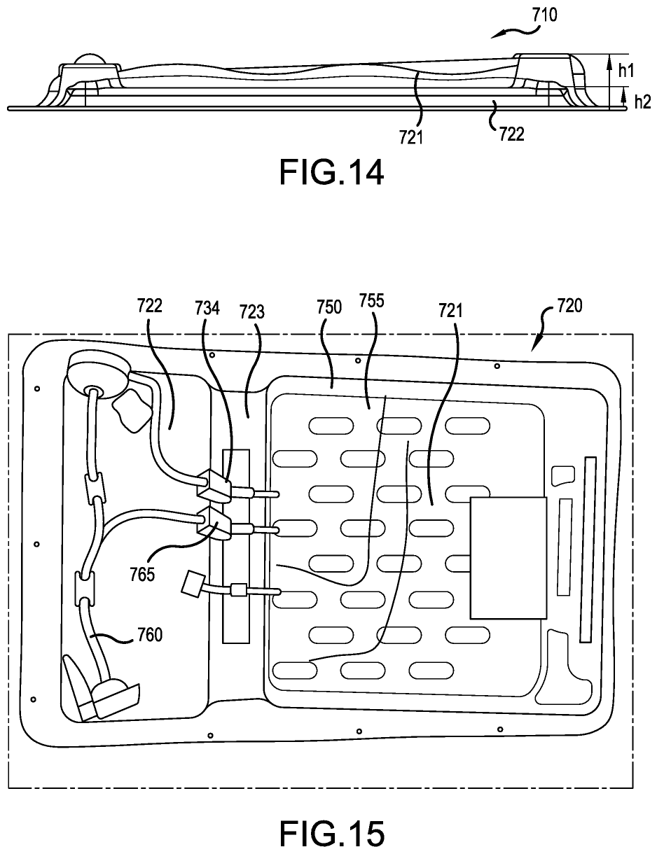

FIG. 14 schematically illustrates an end view of the upper shell 710, according to an exemplary configuration provided herein. As already mentioned above, while only one of the upper shell 710 or the lower shell 720 may be illustrated throughout the figures, the upper shell 710 and the lower shell 720 may be identical. Thus, the description and illustrations of the upper shell 710 are applicable to the lower shell 720 and vice versa.

Referring to FIG. 14, the first compartment 721 of the upper shell 710 may define a deeper compartment than the second compartment 722 of the upper shell 710. That is, the height h1 defining the depth of the first compartment 721 may be greater than the height h2 defining the depth of the second compartment 722. For example, the height h1 may range from about 0.1 inches (about 2.54 millimeters) to about 2 inches (about 5.08 centimeters) and the height h2 may range from about 0.5 inches (about 1.27 centimeters) to about 5 inches (about 12.7 centimeters). In a specific nonlimiting example, the height h1 is about 1.5 inches and the height h2 is about 0.6 inches (about 15.24 millimeters). In an example, there is curvature or wave on a surface of the first compartment 721. For example, the floor 728 is curved or wavy. The curvature or wave may have two functions, among others, including to enhance the air flow and to fill the curvature better of the primary container inside the secondary container than for an almost vertical or flat shell wall and, as a result, prevent significant air pockets from forming. Thus, the primary container may be held in place and better protected. In different examples, the radius of curvature for the curves or the number of curves is relative to the dimensions of the secondary container or primary container or optimized for improving heat transfer. In other examples, the amplitude or frequency of the waves is relative to the dimensions of the secondary container or primary container or optimized for improving heat transfer. The curvature may also inhibit side to side movement of the primary container. Referring back to FIG. 13, it is noted that there is no curvature or wave on the surface of the secondary container 700 when viewed from a side view.

Movement of the frozen product disposed within the secondary container can be limited or prevented by the secondary container. For example, the upper shell 710 and the lower shell 720 of the secondary container 700 can both include wavy surfaces. The wavy surfaces have crests and valleys, where the configuration of crests and valleys limits movement of the primary container and enhances heat transfer between the primary container and a fluid flowing around and through container 700. The wavy surfaces can limit or prevent movement of a frozen primary container. For example, the wavy surfaces can limit or prevent movement of the primary container during cooling (freezing), handling and transport, or heating (thawing) steps. The primary container with a liquid follows the shape formed by the wavy surfaces. Also, the wavy surfaces of the upper shell 710 and the lower shell 720 can have waves that go together with alternating concave convex shapes or crossing waves in same or alternating directions. After freezing the hardened bag cannot move since the wavy surfaces hold it tightly. The secondary container can optionally include other elements to prevent movement of the frozen product within the container. For example, the secondary container can include one or more protuberances that extend from a surface of the secondary container and into a compartment in which a primary container is disposed within. The one or more protuberances can contact the primary container and limit movement of the primary container. The other elements can also aid heat transfer. For example, the one or more protuberances can be made of a material having a thermal conductivity capable of enhancing heat transfer between the secondary container and fluid.

The direction of the wavy surface of the upper shell 710 can be parallel to the direction of the wavy surface of the lower shell 720. Alternatively, the direction of the wavy surface of the upper shell 710 can differ from the direction of the wavy surface of the lower shell 720. For example, the direction of the wavy surface can extend the second wall 725 to the junction 733, where the direction would be perpendicular to direction of the wavy surface shown in FIG. 10 where the direction of the wavy surface extends from the first wall 724 to the third wall 726. In various refinements, the direction of the wavy surface of the upper shell 710 can differ by an angle of 0 degrees (°) or parallel, 2°, 3°, 4°, 5°, 6°, 7°, 8°, 9°, 10°, 11°, 12°, 13°, 14°, 15°, 16°, 17°, 18°, 19°, 20°, 21°, 22°, 23°, 24°, 25°, 26°, 27°, 28°, 29°, 30°, 31°, 32°, 33°, 34°, 35°, 36°, 37°, 38°, 39°, 40°, 41°, 42°, 43°, 44°, 45°, 46°, 47°, 48°, 49°, 50°, 51°, 52°, 53°, 54°, 55°, 56°, 57°, 58°, 59°, 60°, 61°, 62°, 63°, 64°, 65°, 66°, 67°, 68°, 69°, 70°, 71°, 72°, 73°, 74°, 75°, 76°, 77°, 78°, 79°, 80°, 81°, 82°, 83°, 84°, 85°, 86°, 87°, 88°, 89°, or 90° or perpendicular to the direction of the wavy surface of the lower shell 720. In other refinements, the angle that the direction of the wavy surface of the upper shell 710 differs from the wavy surface of the lower shell 720 is a range between any two angles provided above. For example, the range is 1° to 90°, 15° to 90°, 30° to 90°, 45° to 90°, 60° to 90°, 75° to 90°, 1° to 15°, 1° to 30°, 1° to 45°, 1° to 60°, or 1° to 75°. Such angular configurations may further protect the mounting area of bag tubing that is now protected by elastic pad 734 shown in FIG. 15. The crest and valleys of the wavy surfaces of the upper shell 710 can also be the same as or offset from the crest and valleys of the wavy surfaces of the lower shell 710.

The upper and lower shells of the secondary container can optionally include an array of convex and concave elements that can facilitate holding of the bag with product during cooling (freezing), handling and transport and warming (thawing) steps. The concave and convex elements can be located on the opposite walls of the secondary container, that they can match in various ways: concave/convex at the same position (one protrudes into another), or be in offset positions: concave versus concave, convex versus convex, etc. There may be also configurations where the concave/convex elements may be on one wall with another wall being flat.

FIG. 15 illustrates a top planar view of the lower shell 720 of the secondary container 700 (illustrated in assembled form in FIG. 13) with the pinch 734 and a primary container 750. Referring to FIG. 15, the lower shell 720 of the secondary container 700 is illustrated without the upper shell 710. The primary container 750 is to be enclosed between the lower shell 720 and a corresponding upper shell 720 (not shown in FIG. 15) of the secondary container 700. In this example, the primary container 750 includes a bag 755, tubing 760 connected to the bag 755, and an interface 765 between the tubing 760 and the bag 755. As illustrated in FIG. 15, the bag 755 of the primary container 750 may be placed in the first compartment 721, the tubing 760 may be placed in the second compartment 722, and the interface 765 may be placed on the junction 723 and above the pinch 734.

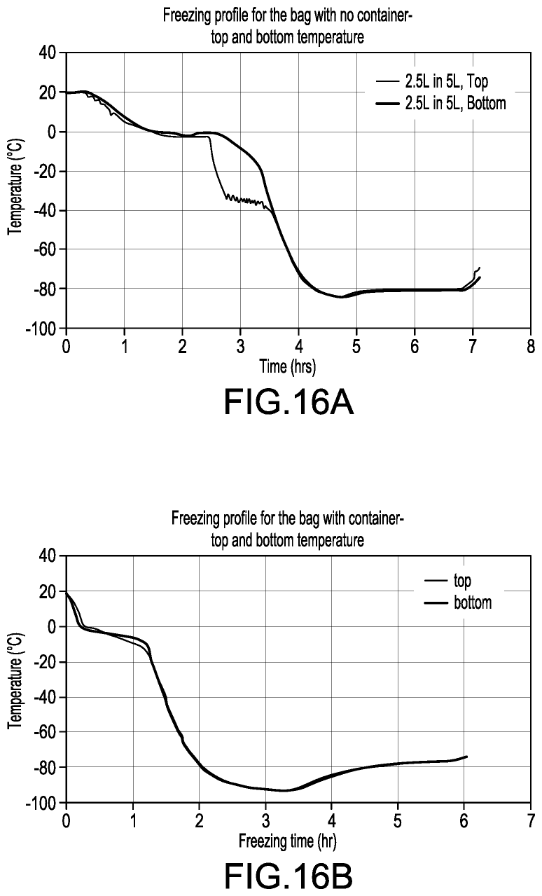

FIG. 16A is a graph illustrating the temperature over time profile of a bag of the primary container without using a secondary container, according to exemplary configurations provided herein. FIG. 16B is a graph illustrating the temperature over time profile of a bag of the primary container while using a secondary container, according to exemplary configurations provided herein.

Referring to FIGS. 16A and 16B, an example of the freezing profile for a 2.5 liter solution in a 5 liter bag of primary container 750 with and without using a secondary container 700, as described throughout the present disclosure. Referring to FIG. 16A, when the primary container 750 is frozen without the secondary container 700, the freezing rate for the top and bottom of the primary container 750 is different and the freezing profile of a top and bottom of the primary container 750 is not even. Referring to FIG. 16B, when the primary container 750 is frozen in the secondary container 700, the freezing rate for the top and bottom of the primary container 750 is the same and the freezing profile of a top and bottom of the primary container 750 is even.

Apertures 727 can face towards a source of fluid flow, such as but not limited to a fan or pump, system duct or system diffuser, orifice of pressurized vessel, wind blowing, or other suitable mechanism that causes flow of fluid. In one nonlimiting example, the fluid is a gas, such as air, nitrogen, or argon. However, it should be appreciated that container 700 and the methods provided herein suitably can be used with fluids that are liquid. Although not wishing to be bound by theory, the fluid flow around and through container 700 can cause areas of high static pressure. The pressure difference between high static pressure areas and the static pressure areas can cause flow of the fluid through the internal volume of secondary container 700. The fluid flow along the curvature or wave on the surface of the first compartment 721 can also generate turbulence or turbulent flow through the apertures 727 such that the fluid enters the internal volume formed by the first compartment 721 and contacts the bag 755. For example, the fluid can contact different areas of the bag 755 such as the top and bottom surfaces of the bag 755 when the primary container 750 is positioned within the secondary container 700. This can allow for heat transfer at different areas of the bag 755 when the fluid is flowing. Fluid flowing through the internal volume of secondary container 700 may flow around, past, and in contact with the inner container disposed therein, and thus can transfer heat to (as in thawing) or from (as in freezing) a product within the inner container, e.g., such as described in greater detail below with reference to FIG. 6. The apertures 727 can enhance the flow of fluid (such as a gas) through the container 700 that in turn improves cooling (e.g., as in freezing) or warming (e.g., as in thawing), or gas or vapor exchange (e.g., as in evaporation, distillation, or drying). The waves of the primary container 750 are exposed to the cooling agent (air or nitrogen gas during freezing), or warming agent (air during thawing) flowing through the secondary container 700. This also can occur if the primary container 750 is processed independently, e.g. if for example, it is placed on a shelf and heat transfer fluid (air, nitrogen) flows around the secondary container 700. The processing usually happens with forced gas flow, for example, under forced convection conditions. In addition and also not wishing to be bound by theory, the wavy surface(s) of the secondary container 700 has a larger heat transfer surface than a flat wall.

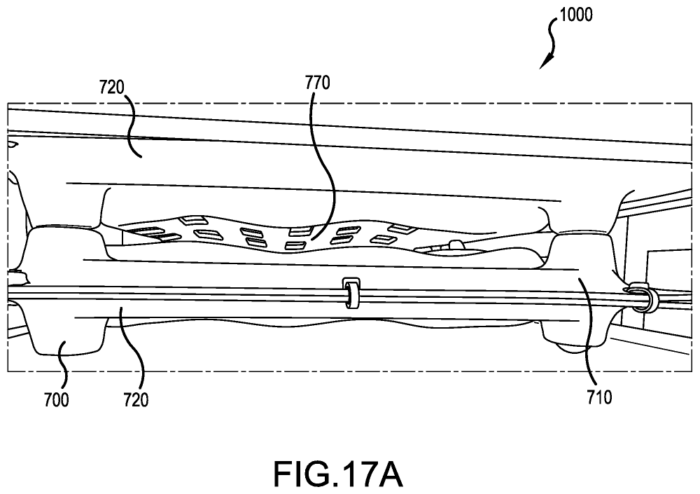

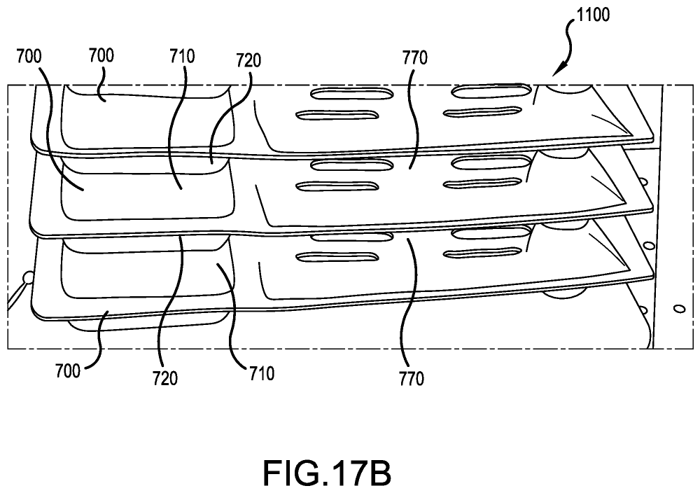

Similar to the arrangement shown in FIG. 5, FIGS. 17A and 17B show secondary containers 700 stacked together 1000,1100. FIG. 17A shows a lower container 720 stacked 1000 on an upper container 710 of secondary container 700. FIG. 17B shows three secondary containers 700 stacked 1100 on top of each other. As shown in FIGS. 17A and 17B, secondary containers 700 stacked together 1000,1100 form internal passages 770 to receive a fluid. The passages 770 are formed between the wavy surface of an upper container 710 of secondary container 700 and a wavy surface of a lower container 720 of a secondary container stacked on the other. The distances between walls of the secondary containers 700 in such passages 770 can be optimized to maximize the heat transfer and/or minimizing flow resistance. In different examples, the size of these passages 770 can range from 0 millimeters (mm) to 500 mm, 1 mm to 350 mm, or 5 mm to 200 mm. In other examples, the ratio of the height to length of the passages ranges from 20:1 to 1:20, 10:1 to 1:10, 5:1 to 1:5, or 2.5:1 to 1:2.5. The distance selection in combination with parameters of the convex/concave surfaces can create flow pattern that controls gas velocities and turbulence toward optimization of freezing and thawing processes. The geometry of entry and exit of the passages 770 that form between the stacked containers can be profiled to further optimize the processes that rely on cooling (cooling and freezing) and heating (warming and thawing) and these entry geometries can be combined with the geometries of concave and convex walls that form the gas passage channels formed between the walls of stacked secondary containers 1000,1100. The geometry of passages 770 can also be optimized to maximize the heat transfer and/or minimizing flow resistance. Although not wishing to be bound by theory, geometries of the passages 770 and/or distances for enhancing heat transfer or minimizing flow resistance may depend on the properties (e.g., phase, temperature, flow rate, etc.) of the fluid or the primary container (e.g., size, type of product, etc.). For example, the properties of air cooling differ from liquid nitrogen cooling.

It should be appreciated that the secondary containers 700 can be used in any suitable process for handling a primary container, e.g., for freezing, thawing, storing, and/or shipping product(s) stored within a primary container. For example, FIG. 6 illustrates a flow of operations in an exemplary method 600 for handling a primary container using a secondary container with flow-through apertures, according to exemplary configurations provided herein. Method 600 illustrated in FIG. 6 includes providing a secondary container (operation 610). The secondary container can be configured such as described elsewhere herein, e.g., container 700 illustrated in FIGS. 10-15. In addition, it should be appreciated that the pressure distributions of FIGS. 7A-7B and demonstrations of the effect of pressure building members as illustrated in FIGS. 8A, 8B, 9A, and 9B, are applicable for use with the secondary container 700 illustrated in FIGS. 10-15.

While various illustrative embodiments of the invention are described above, it will be apparent to one skilled in the art that various changes and modifications can be made therein without departing from the invention. The appended claims are intended to cover all such changes and modifications that fall within the true spirit and scope of the invention.

1.-30. (canceled)

31. A method of freezing a product, the product disposed within an inner container, the method comprising:
disposing, within an internal volume of a secondary container, the inner container having the product disposed therein; and
freezing the product by flowing fluid through the internal volume via apertures defined through at least one sidewall of the secondary container, wherein the fluid is at a temperature selected to freeze the product.

32. The method of claim 31, further comprising:
after freezing the product, storing or shipping the container having the inner container disposed within the internal volume while maintaining the frozen product.

33. The method of claim 31, further comprising:
thawing the frozen product by flowing a second fluid through the internal volume via the apertures, wherein the second fluid is at a temperature selected to thaw the frozen product.

34.-36. (canceled)

37. A container, comprising:
an upper shell, comprising:
an upper shell first compartment comprising a plurality of upper shell first compartment walls and an upper shell first compartment floor, apertures being defined through the upper shell first compartment floor;
an upper shell second compartment comprising a plurality of upper shell second compartment walls and an upper shell second compartment floor;
an upper shell flange extending around the upper shell first compartment and the upper shell second compartment; and
an upper shell junction extending between and separating the upper shell first compartment from the upper shell second compartment; and
a lower shell comprising:
a lower shell first compartment comprising a plurality of lower shell first compartment walls and a lower shell first compartment floor, apertures being defined through the lower shell first compartment floor;
a lower shell second compartment comprising a plurality of lower shell second compartment walls and a lower shell second compartment floor;
a lower shell flange extending around the lower shell first compartment and the lower shell second compartment; and
a lower shell junction extending between and separating the lower shell first compartment from the lower shell second compartment,
wherein the upper shell flange and the lower shell flange contact one another to define an internal volume,
wherein the apertures defined through the upper shell first compartment floor and the apertures defined through the lower shell first compartment floor are configured such that fluid flows through the internal volume through those apertures.

38. The container of claim 37, further comprising one or more pinches positioned attached to at least one of the upper shell junction and the lower shell junction.

39. The container of claim 37, further comprising one or more holes and one or more counterpart holes extending through the upper shell flange and the lower shell flange, respectively.

40. (canceled)

41. The container of claim 37, wherein at least one of the upper shell first compartment floor and the lower shell first compartment floor is wavy or flat.

42. (canceled)

43. The container of claim 37, wherein the upper shell first compartment has a greater depth than upper shell second compartment and the lower shell first compartment has a greater depth than the lower shell second compartment.

44.-51. (canceled)

52. The container of claim 37, wherein the fluid flows through a portion of the internal volume within the upper shell at a rate that is about the same as through a portion of the internal volume within the lower shell.

53. (canceled)

54. The container of claim 37, further comprising an inner container disposed within the internal volume.

55. (canceled)

56. (canceled)

57. The container of claim 37, wherein the fluid is at a temperature selected to thaw a frozen solid.

58. (canceled)

59. The container of claim 54, wherein the fluid is at a temperature selected to freeze a liquid present in a product present in the inner container.

60. (canceled)

61. The container of claim 54, wherein the inner container comprises a bag, tubing, and an interface between the bag and the tubing.

62. The container of claim 61, wherein the inner container bag is disposed between the upper shell first compartment and the lower shell first compartment.

63. The container of claim 61, wherein the inner container tubing is disposed between the upper shell second compartment and the lower shell second compartment.

64. The container of claim 61, wherein the inner container interface is disposed between the upper shell junction and the lower shell junction.

65. The container of claim 37, wherein:
the upper shell first compartment, the upper shell second compartment, the upper shell flange, and the upper shell junction are of unitary construction,
the lower shell first compartment, the lower shell second compartment, the lower shell flange, and the lower shell junction are of unitary construction,
the upper shell flange and the lower shell flange are separable from one another.

66. The container of claim 37, wherein the upper shell is identical to the lower shell.

67. The container of claim 37, wherein, in response to freezing an inner container disposed within the container, a freezing rate for a top and bottom of the inner container is the same and a freezing profile a top and bottom of the inner container is even.

68. (canceled)

69. A kit comprising:
an upper shell, comprising:
an upper shell first compartment comprising a plurality of upper shell first compartment walls and an upper shell first compartment floor, apertures being defined through the upper shell first compartment floor;
an upper shell second compartment comprising a plurality of upper shell second compartment walls and an upper shell second compartment floor;
an upper shell flange extending around the upper shell first compartment and the upper shell second compartment; and
an upper shell junction extending between and separating the upper shell first compartment from the upper shell second compartment; and
a lower shell comprising:
a lower shell first compartment comprising a plurality of lower shell first compartment walls and a lower shell first compartment floor, apertures being defined through the lower shell first compartment floor;
a lower shell second compartment comprising a plurality of lower shell second compartment walls and a lower shell second compartment floor;
a lower shell flange extending around the lower shell first compartment and the lower shell second compartment; and
a lower shell junction extending between and separating the lower shell first compartment from the lower shell second compartment,
wherein the upper shell flange and the lower shell flange are configured to be brought into contact with one another to define an internal volume.
